(19) United States
(12) Patent Application Publication
(10) Pub. No.: US 2008/0120784 A1
(43) Pub. Date: May 29, 2008

(54) SMART BED SYSTEM AND APPARATUS

(75) Inventors: Adrian F. Warner, Delafield, WI (US); Michael T. Suchecki, Waukesha, WI (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/564,051

(22) Filed: Nov. 28, 2006

(51) Int. Cl. A47C 31/00; A61B 5/00; A61B 5/02; G06F 3/033; G06F 3/041

(52) U.S. Cl. 5/658; 600/300; 345/161; 345/163; 345/167; 345/168; 345/173; 345/184; 340/825.22

(57) ABSTRACT

A smart bed apparatus adapted to retain a patient and a smart bed system are disclosed herein. The smart bed apparatus includes a smart bed computer, and a patient monitoring device operatively connected to the smart bed computer. The patient monitoring device is incorporated with the smart bed such that the smart bed is not restricted by any wires adapted to couple the patient monitoring device with the patient. The smart bed system includes a server coupled with the smart bed computer. The server is configured to store any patient data pertaining to the patient. The patient data can be selectively transferred from the server to the smart bed computer such that the patient data is accessible directly from the smart bed.

### FIELD OF THE INVENTION

This disclosure relates to a smart bed system and apparatus adapted for automated patient monitoring.

### BACKGROUND OF THE INVENTION

Hospitals may implement a variety of different automated patient monitoring devices for purposes of monitoring patients and recording the resultant data. Examples of such automated patient monitoring devices include electrocardiograph (ECG) devices, pulse oximeter devices, blood-pressure monitoring devices, etc. A plurality of wires couple the automated patient monitoring devices with the patient. The automated patient monitoring devices are typically disposed on stands or carts adjacent to a patient's hospital bed.

The problem is that the wires can impede or restrict patient motion and may therefore encourage patients to minimize their activity level. Prolonged patient inactivity is linked to medical conditions such as thrombosis and muscle atrophy. Another problem with the wires pertains to hospital beds that are designed to be mobile such that patients can be transported without getting up. The wires connecting the automated patient monitoring devices with the patient can interfere with the process of transporting mobile hospital beds containing a patient. If the wires are disconnected from the patient or the automated patient monitoring devices in order to facilitate the transportation of the mobile hospital beds, the patient cannot be monitored during such transportation.

### BRIEF DESCRIPTION OF THE INVENTION

The above-mentioned shortcomings, disadvantages and problems are addressed herein which will be understood by reading and understanding the following specification.

In an embodiment, a smart bed adapted to accommodate a patient includes a smart bed computer, and a patient monitoring device operatively connected to the smart bed computer. The patient monitoring device is incorporated with the smart bed such that the smart bed is not restricted by any wires adapted to couple the patient monitoring device with the patient.

In another embodiment, a smart bed adapted to accommodate a patient includes a smart bed computer, a patient monitoring device operatively connected to the smart bed computer, and a plurality of patient sensors operatively connected to the patient monitoring device. The smart bed also includes a bed monitoring device operatively connected to the smart bed computer, and a plurality of bed sensors operatively connected to the bed monitoring device. The patient monitoring device is incorporated with the smart bed such that the smart bed is not restricted by any wires adapted to couple the patient monitoring device with the plurality of patient sensors.

In yet another embodiment, a smart bed system includes a smart bed adapted to accommodate a patient, said smart bed includes a smart bed computer, and a patient monitoring device operatively connected to the smart bed computer. The patient monitoring device is incorporated with the smart bed such that the smart bed is not restricted by any wires adapted to couple the patient monitoring device with the patient. The smart bed system also includes a server coupled with the smart bed computer. The server is configured to store any patient data pertaining to the patient. The patient data can be selectively transferred from the server to the smart bed computer such that said patient data is accessible directly from the smart bed.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of a smart bed system in accordance with an embodiment;

FIG. 2 is a block diagram illustrating an algorithm in accordance with an embodiment;

FIG. 3 is a schematic diagram illustrating a smart bed coupled with a plurality of input devices in accordance with an embodiment;

FIG. 4 is a schematic diagram illustrating a smart bed coupled with a plurality of output devices in accordance with an embodiment; and

FIG. 5 is a block diagram illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

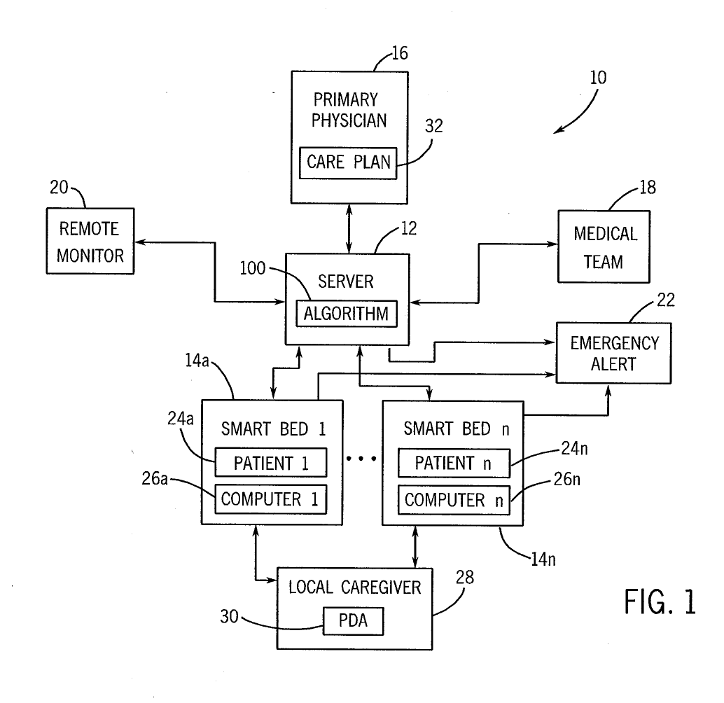

Referring to FIG. 1, a smart bed system 10 in accordance with an embodiment is shown. The smart bed system 10 includes a server 12 operatively connected to one or more smart beds 14a-14n. For purposes of the present invention, a “server” is defined to include any remotely accessible device having a processor and a storage medium. It should be appreciated that the smart bed system 10 may include multiple servers, and that the schematically depicted server 12 may represent a plurality of servers.

The server 12 receives data from and transmits data to a plurality of different sources for purposes such as, for example, continuously monitoring the patient; directly addressing or directing others to address patient needs; implementing precautionary measures to ensure the patient is optimally cared for; and conveying relevant patient data to an individual or a team for purposes of obtaining a diagnosis or developing a care plan. In a non-limiting manner, these sources may include the smart beds 14a-14n; a primary physician 16; a medical team 18; a remote monitor 20; and/or an emergency alert device 22.

Each of the smart beds 14a-14n is respectively adapted to retain one of the patients 24a-24n. The smart beds 14a-14n each include a computer 26a-26n that is coupled to the server 12 in a conventional manner. It should be appreciated that each of the smart beds 14a-14n may include multiple computers, and that the schematically depicted computers 26a-26n may each represent a plurality of computers. A local caregiver 28 such as a nurse can implement the smart bed computers 26a-26n for transferring data to or retrieving data from the server 12 in order to optimally meet the needs of the patients. According to one embodiment, the local caregiver 28 may implement a personal digital assistant (PDA) 30 to access the computers 26a-26n. Alternatively, the local caregiver 28 can access the computers 26a-26n in any known manner such as with a keyboard, mouse, touch screen, etc. According to another embodiment, the smart beds 14a-14n may be configured to automatically transfer data to or retrieve data from the server 12 as will be described in detail hereinafter.

The primary physician 16 may either directly or remotely extract data from and input data into the server 12. An exemplary interaction between the primary physician 16 and the server 12 may include the following. The primary physician 16 initially extracts information from the server 12 such as the medical history and current vital statistics for patient 24a. After obtaining and evaluating this information from the server 12, the primary physician 16 develops a care plan 32 adapted to optimally treat the patient 24a and inputs the care plan 32 into the server 12. The care plan 32 is accessible to the local caregiver 28 via the smart bed 26a such that the local caregiver 28 can implement the primary physician's care plan 32. During the course of treatment, the primary physician 16 can extract additional information such as any subsequently acquired patient data in order to evaluate patient progress and to adjust the care plan 32. Accordingly, the smart bed system 10 allows the primary physician to analyze and treat multiple patients from a remote location.

The medical team 18 can either directly or remotely extract data from and input data into the server 12. As the server 12 is remotely accessible, the medical team 18 may comprise members from distant geographical regions. Accordingly, specialists from all over the world can be virtually brought together to as part of the medical team 18 in order to evaluate and treat the patients 24a-24n. An exemplary interaction between the medical team 18 and the server 12 may include the following. The medical team 18 initially extracts information from the server 12 such as the patient's medical history, initial diagnosis, medical reports, care plan 32, etc. Thereafter, the medical team members collectively evaluate the information from the server 12 in order provide additional feedback. This feedback may then be input into the server 12 for further consideration by the primary physician 16 and so that the local caregiver 28 can carry out any instructions. Accordingly, the smart bed system 10 supports team based medicine by providing a plurality of remotely located team members the opportunity to review a common collection of patient data for the purpose of diagnosing and/or treating the patient. As the server 12 is directly accessible, the smart bed system 10 also supports local team based medicine.

The remote monitor 20 is generally an individual or group of individuals that remotely access the server 12 in order to observe or monitor data from one or more of the patients 24a-24n. Accordingly, the remote monitor 20 can provide an additional layer of protection for the patients 24a-24n. In the event that the remote monitor 20 observes problematic patient data, the remote monitor 20 can alert the primary physician 16 and/or the local caregiver 28 for further analyses or follow-up. The remote monitor 20 may also remotely trigger the emergency alert device 22, which is described in detail hereinafter, if problematic patient data is observed.

The emergency alert device 22 may be triggered manually from the server 12 or any of the smart beds 14a-14n, or may be triggered automatically by the algorithm 100 described in detail hereinafter. The emergency alert device includes an alarm system adapted to alert the primary physician 16, the local caregiver 28 and/or any other staff members in order to quickly address a medical emergency. The emergency alert device 22 may be configured to alert local personnel with conventional audio (e.g., a siren or verbal warning) and/or visual (e.g., a flashing light) feedback, and may also be configured to alert remotely located personnel such as by calling a cell phone or pager, or by sending a text message.

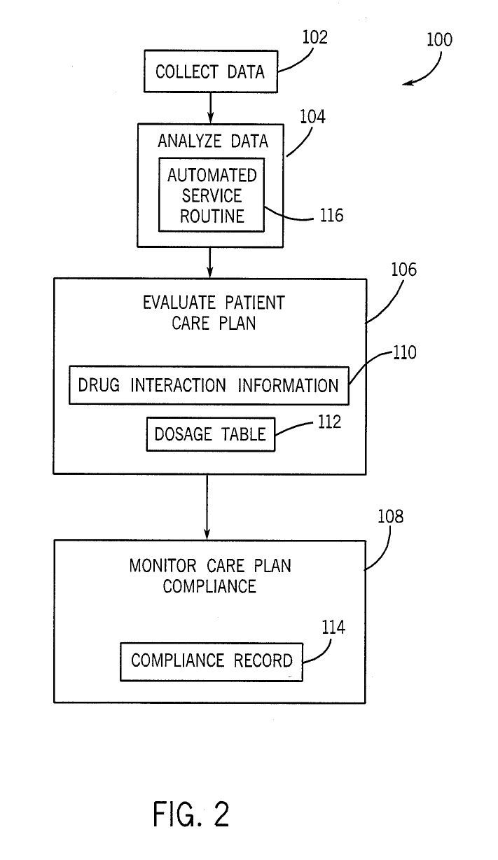

Referring to FIG. 2, a block diagram illustrating the algorithm 100 is shown. The individual blocks of FIG. 2 represent steps that may be performed in accordance with the algorithm 100. According to one embodiment, the algorithm 100 may be stored on the server 12 (shown in FIG. 1) of the smart bed system 10 (shown in FIG. 1), and can be generally continuously operated for purposes such as providing additional protection for the patients 24a-24n (shown in FIG. 1), improving the level of care provided to the patients 24a-24n, and minimizing labor by automating processes that would otherwise be manually performed. According to another embodiment, the algorithm 100 may be stored on each of the smart bed computers 26a-26n (shown in FIG. 1) individually.

At step 102, the algorithm 100 is configured to automatically collect patient data. The patient data can be collected from that which is stored directly on the server 12 (shown in FIG. 1) and from any source coupled to the server 12. As an example, the algorithm 100 may be implemented to retrieve the vital statistics that are potentially indicative of a cardiovascular disease (e.g., blood pressure, heart rate, etc.) from the smart bed of a particular patient. Thereafter, the retrieved data can be automatically compiled by the algorithm 100 in a convenient manner such as a plot, graph, chart, or medical record. The compiled data is then presentable to any local or remote caregiver in the form of a medical report. It should be appreciated that the specific type of data collected and the format in which it is presented are selectable in order to optimally meet the needs of a particular patient.

As another example, at step 102 the algorithm 100 may be implemented to retrieve all billing data which may include, for example, a listing of the services provided and their associated cost; the duration of the patient's hospitalization; the patient's insurance provider and type of coverage; etc. Thereafter, the retrieved data can automatically be compiled by the algorithm 100 in the form of one or more invoices that include an itemized listing of expenses. As an example, if the patient's medical plan includes a co-pay, a first invoice covering the amount of the co-pay can be automatically prepared and sent to the patient, and a second invoice covering the remainder of the expenses can be automatically prepared and sent to the patient's insurance provider.

At step 104, the algorithm 100 is configured to automatically analyze patient data. As an example, the algorithm 100 can be configured to identify or flag any patient data that falls outside a predefined range, and to initiate an appropriate response to such flagged data. Exemplary responses may include alerting a physician or other hospital staff member, scheduling a medical team case review meeting, and/or automatically initiating a course of action. The most appropriate response may depend upon the specific type of data that has been flagged and/or the amount by which the flagged data falls outside the predefined range. The automatic initiation of a course of action is a feature enabled by the smart bed system 10 (shown in FIG. 1) as will be described in detail hereinafter. It should be appreciated that the specific type of data analyzed and the manner in which it is analyzed are selectable in order to optimally meet the needs of a particular patient.

Step 104 may also include an automated service routine 116. The service routine 1 16 may, for example, be configured to monitor smart bed system 10 operation and identify any fault conditions so that any service needs can be proactively met. The fault conditions are selectable and may, for example, be predicated on the electrical, mechanical and software functionality of the smart bed system 10. According to one example, the service routine 116 can record the number of hours of “in use” smart bed operation and forward the recorded information (including any recorded failure events) to a service provider system for analysis and fault determination. The automated service routine 116 may also record maintenance that has been carried out on the smart bed system 10, indicate to a caregiver or service provider when routine maintenance is required, and indicate “out of service” on any smart bed that requires service in order to prevent additional patients from being admitted. If a patient occupies a smart bed that requires service, the automated service routine 116 may assign the patient a new smart bed so that the patient can be cared for while the original smart bed is serviced.

At step 106, the algorithm 100 is configured to evaluate the patient care plan 32 (shown in FIG. 1) in order to provide an additional layer of protection for the patients 24a-24n (shown in FIG. 1). The algorithm 100 may, for example, be adapted to compare the care plan 32 with a patient's medical history to verify that the patient is not allergic or overly sensitive to any prescribed medicine. According to one embodiment, the algorithm 100 may have access to a database containing drug interaction information 110. The algorithm 100 can compare the drug interaction information 110 with the care plan 32 to ensure the patient is not prescribed and/or administered a potentially harmful combination of medications. According to another embodiment, the algorithm 100 may have access to a recommended dosage table 1 12 providing dosage ranges for a variety of different medications based on, for example, the patient's sex, weight, age, etc. The algorithm 100 may compare the care plan 32 with the recommended dosage table 112 in order to prevent an overdose. The drug interaction information 110 and the recommended dosage table 112 may be stored directly on the server 12 (shown in FIG. 1) or on any system accessible by the server 12. If any potential problems with the care plan 32 are identified, the algorithm 100 can initiate an appropriate response to such problems including those responses previously described with respect to step 104.

At step 108, the algorithm 100 is configured to monitor care plan 32 (shown in FIG. 1) compliance. As an example, the performance of any activities specified by the care plan 32 and the time at which such activities were performed can be either manually or automatically saved in the form of a compliance record 114. The algorithm 100 can then compare the care plan 32 with the compliance record 114 to ensure the care plan 32 is properly executed. If a deviation from the care plan 32 is identified, the algorithm 100 can convey such information in an appropriate manner in order to minimize any effects of the deviation and to prevent future deviations.

Having described the smart bed system 10 (shown in FIG. 1) in accordance with several exemplary embodiments, the smart bed 14a (shown in FIG. 1) will now be described in more detail. It should be appreciated that the smart bed 14a is described in the following section for illustrative purposes, and that the other smart beds 14b-14n (shown in FIG. 1) may be similarly configured. According to an alternate embodiment, the smart beds 14a-14n may be individually configured to provide specialized care such as, for example, one or more smart beds that are specially adapted for burn care, cardiac rehabilitation, or intensive care.

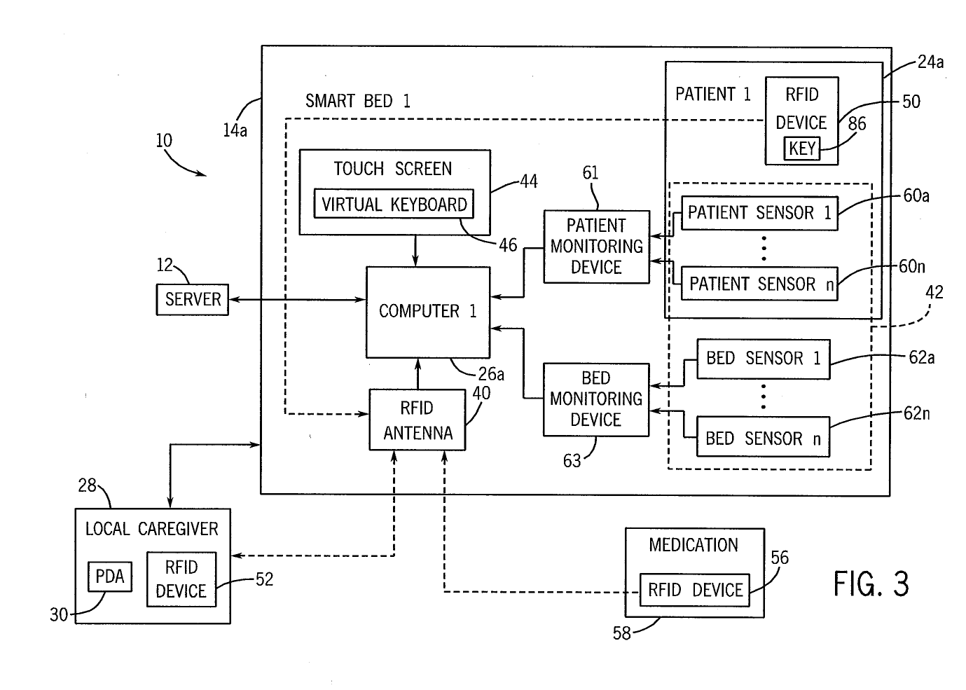

As shown in FIG. 3, the smart bed computer 26a can be coupled to the server 12 and to a plurality of different input devices. The input devices may include, for example, a wireless communication device (e.g., the radio frequency identification (RFID) antenna 40), a plurality of sensors 42, a touch screen 44 having a virtual keyboard 46, and one or more monitoring devices (e.g., the patient monitoring device 61 and the bed monitoring device 63). For purposes of this disclosure, the term “monitoring” may be defined to include, in a non-limiting manner, acts such as recording, observing, evaluating, identifying, etc.

The RFID antenna 40, any of the sensors 42a, and/or the touch screen 44 may be configured to operate both as an input device and an output device (i.e., an I/O device), however, they will be described as being an input devices for purposes of this disclosure. The touch screen 44 is optional and may alternatively include other known input devices such as a keyboard, mouse, touch pad, joystick, remote control (either wireless or with a wire), track ball, Marquette trim knob, etc. According to one embodiment, the smart bed computer 26a is wirelessly coupled to the server 12, and the RFID antenna 40 is mounted directly to the smart bed 14a in order to minimize the number of external cables restricting smart bed motion.

The wireless communication device will hereinafter be described as the RFID antenna 40, however, other wireless communication devices such as, for example, a bar code reader may also be envisioned. The RFID antenna 40 may receive input from a RFID device 50 secured to the patient 24a in a conventional manner such as with a wristband. During admission, a patient's RFID device 50 can be programmed to include a wide range of information including patient identification information (e.g., sex, age, height, weight), medical information (e.g., medical history, allergies, dietary restrictions), billing information (e.g., insurance carrier), etc. Thereafter, additional patient information that has been collected during the course of treatment can be added to the RFID device 50. When the RFID device 50 is in sufficiently close proximity to the RFID antenna 40, any information programmed into the RFID device 50 can be downloaded onto the smart bed computer 26a and transferred to the server 12. The RFID device 50 may also include an encryption device 86 as described in detail hereinafter.

The RFID antenna 40 may also receive input from an RFID device 52 secured to the local caregiver 28 or other hospital personnel, and from one or more RFID devices 56 secured to objects such as an IV bag or bottle containing medication 58. The RFID device 52 secured to the local caregiver 28 may be programmed to include, for example, the local caregiver's identity and occupation. Therefore, whenever the local caregiver 28 wearing the RFID device 52 comes into sufficiently close proximity to the RFID antenna 40, the local caregiver's identity and the time can be automatically recorded by the computer 26a and transferred to the server 12. The local caregiver 28 can also manually input additional information directly into the smart bed computer 26a via the PDA 30, the touch screen 44, a dedicated remote controller (not shown), or any other input device. This additional information may include the purpose of the visitation, any findings, any procedures administered, etc.

The RFID device 56 secured to an object such as an IV bag or bottle containing medication 58 may be programmed to include, for example, the type and quantity of medication. Therefore, when the medication 58 is administered to the patient 24a, and the RFID device 56 is in sufficiently close proximity to the RFID antenna 40, information such as the type and quantity of medication administered and the time at which it was administered can be automatically calculated and recorded by the computer 26a and transferred to the server 12. The RFID device 56 is re-programmable so that, for example, after a predetermined portion of the medication 58 has been administered, the RFID device 56 can be reprogrammed to reflect the remaining quantity of medication 58 in the IV bag or bottle. The remaining quantity of medication 58 can also be provided to the master materials management scheduler for the facility such that an accurate count can be maintained and so that additional supplies can be ordered in a timely manner.

Other RFID antennas (not shown) associated with the other smart beds 14b-14n (shown in FIG. 1) can also receive input from a given patient's RFID device. Accordingly, the smart bed system 10 (shown in FIG. 1) may be implemented as a patient locating and tracking device. More precisely, hospital personnel can access the server 12 in order to determine which of the smart beds 14a-1 4n are receiving a signal from the RFID device of the patient to be located. A hospital may also include additional RFID antennas (not shown) for the purpose of more thoroughly locating and tracking its patients.

The plurality of sensors 42 include a first group of sensors attached directly to the patient (patient sensors) 60a-60n and a second group of sensors attached to the smart bed (bed sensors) 62a-62n. The patient sensors 60a-60n may include both those attached to a patient in an invasive manner and those attached in a non-invasive manner. For purposes of this disclosure, “bed sensors” are defined to include sensors disposed within or attached to the blankets, sheets, and pillows.

In a non-limiting manner, the patient sensors 60a-60n may include devices adapted to measure patient motion, weight, temperature, blood pressure, blood glucose level, pulse, heart rate, etc. Advantageously, the incorporation of the patient sensors 60a-60 n allow the patient 24a to be generally continuously monitored. Sensor data can be recorded by the computer 26a and transferred to the server 12. According to one embodiment, the sensor data can be implemented by the algorithm 100 (shown in FIG. 2) for automated analysis as described with respect to step 104 (shown in FIG. 2).

The patient sensors 60a-60n may be coupled with the patient monitoring device 61 which is preferably attached to or incorporated into the smart bed 14a. As an example, an electrocardiogram (ECG) device (not shown) can be incorporated into the smart bed 14a and coupled to one or more patient sensors 60a-60n in order to measure heart rate and pulse. By either attaching the patient monitoring device 61 directly to the smart bed 14a or by incorporating the monitoring device 61 -into the smart bed 14a, the smart bed 14a can be designed such that there are fewer external wires restricting bed motion. Therefore, the patient 24a can be conveniently transported without restriction while remaining in beds and the patient 24a can also be continuously monitored during such transportation. It should be appreciated that the schematically depicted patient monitoring device 61 may represent a plurality of patient monitoring devices. In a non-limiting manner, the patient monitoring device 61 may include devices such as the previously mentioned ECG device, a blood pressure monitoring device, a body temperature monitoring device, pulse oximeter device, an electromyogram (EMG) device, an electroencephogram (EEG) device, etc.

According to one embodiment, one or more patient monitoring devices 61 may be removeably attachable to the smart bed 14a such as with a cartridge type attachment. Accordingly, the smart bed 14a can be set up to include only those monitoring devices 61 that are necessary for a particular patient. If the patient 24a chooses to get up from the smart bed 14a, the monitoring devices 61 can be removed and transported along with the patient such as on a mobile stand or rack. In this manner, the patient 24a is less restricted by wires, and the patient 24a can walk around while being continuously monitored.

In a non-limiting manner, the bed sensors 62a-62n may include one or more pressure sensors and/or mass sensors. Pressure sensors may be used to identify the presence of the patient 24a within the smart bed 14a, to monitor patient movement into and out of the smart bed 14a, and to monitor patient movement within the smart bed 14a. As an example, if the pressure sensors indicate excessive patient inactivity it may become necessary to implement precautionary measures in order to prevent thrombosis. Mass sensors may be implemented to monitor patient weight loss and gain. Patient weight loss or gain may be used, for example, in combination with fluid administration and excretion data in order to estimate kidney function.

According to another embodiment, the bed sensors 62a-62n may include a conductance sensor array adapted to identify the presence of bodily fluids that come into contact with the smart bed 14a. The identification of such fluids can be conveyed to an appropriate hospital staff member in a conventional manner. The use of a sensor adapted to measure salinity allows the smart bed system 10 to distinguish between blood and urine. The smart bed system 10 can also identify the location of the bodily fluid relative to the patient 24a as another technique for distinguishing between blood and urine. For example, if the bodily fluid is in close proximity to the patient's groin, the bodily fluid may be assumed to comprise urine. Similarly, if the bodily fluid is in close proximity to a documented wound, the bodily fluid may be assumed to comprise blood. This information can allow for the early detection of bleeding and infection weepage, and is particularly helpful for unconscious patients.

The bed sensors 62a-62n may be coupled with the bed monitoring device 63 which is preferably attached to or incorporated into the smart bed 14a. By either attaching the bed monitoring device 63 directly to the smart bed 14a or by incorporating the monitoring device 63 into the smart bed 14a, the smart bed 14a can be designed such that there are fewer external wires restricting bed motion. Therefore, the patient 24a can be conveniently transported without restriction while remaining in bed, and the smart bed 14a can also be continuously monitored during such transportation. It should be appreciated that the schematically depicted bed monitoring device 63 may represent a plurality of bed monitoring devices. In a non-limiting manner, the bed monitoring device 63 may include devices adapted to monitor pressure, mass, temperature, fluid presence, etc. According to one embodiment, one or more bed monitoring devices 63 may be removeably attachable to the smart bed 14a such as with a cartridge type attachment in a manner similar to that described hereinabove with respect to the patient monitoring devices 61.

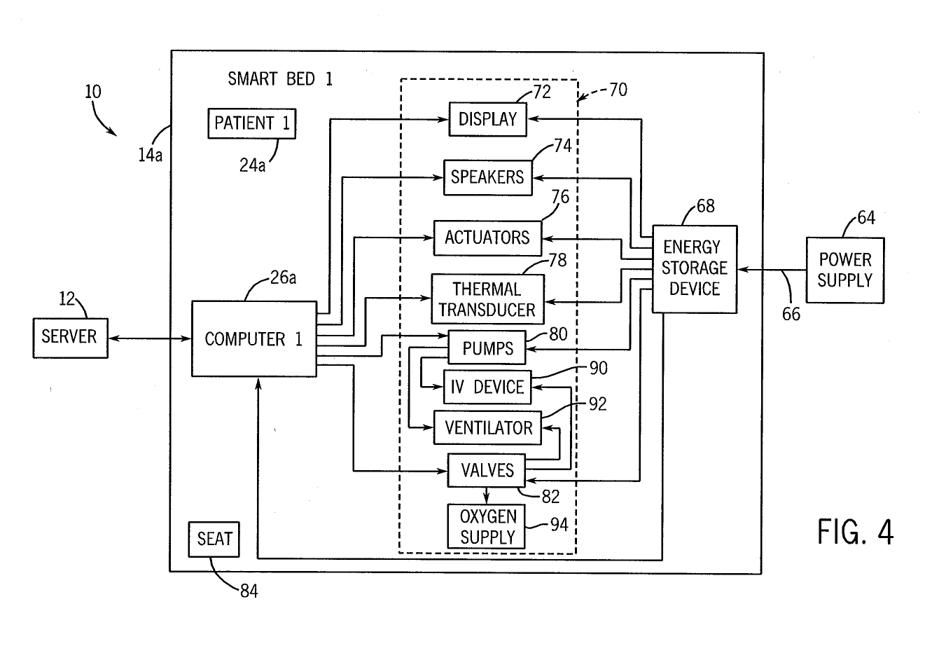

Referring to FIG. 4, the smart bed 14a can be powered by an external power supply 64 such as an electrical outlet (not shown) via the power cable 66. According to one embodiment, the smart bed 14a includes an energy storage device 68 such as a re-chargeable battery that is adapted to store energy from the external power supply 64. Advantageously, this embodiment allows the smart bed 14a to be unplugged from the external power supply 64 and powered by the storage device 68 so that the smart bed 14a remains fully operational without restriction from the power cable 66.

The smart bed 14a can include a plurality of output devices 70 configured to at least partially automate the process of caring for the patient 24a, and to convenience both the patient 24a and the hospital staff members. For purposes of the present invention, the phrase “caring for a patient” is defined to include, in a non-limiting manner, acts such as treating a patient, assisting a patient, meeting any patient needs or preferences, comforting the patient, etc. The output devices 70 may, for example, be positioned within the smart bed 14a (including any sheets, blankets, pillows, etc.), attached to the smart bed 14a, or integrally formed as part of the smart bed 14a. In a non-limiting manner, the output devices 70 can include a display 72, speakers 74, actuators 76, thermal transducers 78, pumps 80, valves 82, etc. According to an embodiment, the emergency alert 22 (shown in FIG. 1) can be incorporated into the smart bed 14a such that it would also be included as one of the output devices 70. According to another embodiment, the output devices 70 may include lights (not shown) that may be operated to convenience the patient or turned off to create an environment conducive to rest.

The display 72 may optionally incorporate the touch screen 44 (shown in FIG. 3) such that the display 72 becomes an I/O device, however, for purposes of this disclosure the display 72 will be described as an output device. The actuators 76 may include known devices such as electrical or hydraulic servos adapted to selectively adjust and control the position of the smart bed 14a. For example, the actuators 76 may raise and lower the entire bed, the head of the bed, and/or the foot of the bed. The thermal transducers 78 may be disposed in the smart bed 14a (including any blankets, sheets, pillows, etc.) to selectively raise or lower the temperature. The pumps 80 may be used to transfer liquids or gasses such as for the purpose of operating a powered IV device. The valves 82 may be operated to regulate fluid flow for devices such as IV systems, oxygen supply systems, and anesthesia systems.

According to one embodiment, the smart bed system 10 may be configured to convenience the patient 24a by automatically adjusting the smart bed 14a in a personalized manner. As an example, information pertaining to a patient's short stature or relative weakness can be programmed onto the patient's RFID device 50 (shown in FIG. 3) or directly input into the smart bed computer 26a. This information can be used to assist a patient that may otherwise have trouble getting into or out of the smart bed 14a. More precisely, when the patient 24a approaches the smart bed 14a, the smart bed's RFID antenna 40 (shown in FIG. 3) can sense the patient's RFID device 50. Thereafter, the smart bed 14a can transfer power to one or more of the actuators 76 in order to automatically lower the smart bed 14a and thereby facilitate entry. Once there is an indication that the patient 24a is in bed (e.g., as indicated by feedback from pressure sensors in the smart bed), the smart bed 14a can automatically raise to a predetermined level. The patient 24a can input a command via the touch screen 44 (shown in FIG. 3) in order to lower the smart bed 14a and more conveniently exit the smart bed 14a.

According to another embodiment, the smart bed 14a may be configured to automatically adjust in a manner adapted to convenience the local caregiver 28 (shown in FIG. 3) or other hospital staff members. For example, information pertaining to a given staff member's physical characteristics, limitations, and/or preferences can be programmed into each staff member's RFID device or input directly into the smart bed 14a. Thereafter, the smart bed 14a can automatically adjust in an optimally ergonomic manner for each staff member. The smart bed 14a may also, for example, be configured to automatically adjust in a manner adapted to facilitate the performance of a specified procedure. The smart bed 14a can also include a seat 84 adapted to further convenience the local caregiver 28 (shown in FIG. 3) or other hospital staff members. The seat 84 can be attached to the smart bed 14a in a conventional manner or can be integrally incorporated into the design of the smart bed 14a. The seat 84 may be adjustable in an up/down direction, an in/out direction, and may also be translatable around the periphery of the smart bed 14a such that a caregiver can remain seated in an optimally ergonomic manner while attending to the patient 24a. According to one embodiment, the seat 84 can be extended from the smart bed 14a during use and is otherwise retracted within or under the smart bed 14a.

According to another embodiment, the smart bed 14a may be configured to implement the thermal transducers 78 in order to care for the patient 24a. As an example, if the patient 24a is suffering from a fever, the primary physician 16 (shown in FIG. 1) may specify in the care plan 32 (shown in FIG. 1) that the patient 24a be subjected to a low temperature environment. The smart bed system 10 can control the thermal transducers 78 in response to such a command in order to produce a low temperature environment within the smart bed 14a. Conversely, if a patient is suffering from hypothermia, the smart bed system 10 can raise temperature of the smart bed 14a. Data from the sensors 42 (shown in FIG. 3) indicating that the patient's body temperature is excessively high or low may also be used to trigger the thermal transducers 78 and thereby automatically comfort or care for the patient 24a.

According to another embodiment, the smart bed 14a may be configured to implement the pumps 80 and/or the valves 82 in order to care for and protect the patient 24a. As an example, the pumps 80 and/or valves 82 may be automatically operated to transfer IV fluid at a predetermined rate in accordance with instructions from the care plan 32 (shown in FIG. 1). As another example, if the smart bed computer 26a receives an indication (e.g., from the RFID antenna 40) that the patient 24a is to receive an IV medication that is inconsistent with the care plan 32 (shown in FIG. 1) or the patient's medical history (e.g., allergy), the smart bed 14a can stop the pump 80 driving the IV system in order to prevent the inconsistent medication from being administered. Accordingly, the smart bed system 10 can automatically initiate precautionary measures in order to protect the patient 24a.

According to another embodiment, the smart bed 14a may be configured to implement the display 72 and speakers 74 to entertain and care for the patient 24a. As an example, the patient 24a may directly request or the care plan 32 (shown in FIG. 1) may dictate the operation of the smart bed display 72 and speakers 74 to entertain or stimulate the patient. In a non-limiting manner, the display 72 and speakers 74 may provide visual and/or audio stimulation such as television, movies, music, Internet access, video games, etc. As another example, if the care plan 32 indicates the patient 24a should rest, the speakers 74 can incorporate noise cancellation technology to provide a quiet environment that is conducive to resting or sleeping. The noise cancellation technology may be particularly helpful to patients that share a single room with other potentially noisy patients.

According to another embodiment, the smart bed 14a may include one or more patient care devices operatively connected to one or more of the output devices 70 in order to facilitate the process of caring for the patient 24a. In a non-limiting manner, the patient care devices may include an IV device 90, a ventilator 92, an oxygen supply device 94, or any other known device adapted to care for a patient. According to one exemplary embodiment, the IV device 90 and the ventilator 92 are both operatively connected to one of the pumps 80 and to one of the valves 82. In this manner, the pumps 80 can power the IV device 90 and ventilator 92, and the valves 82 can control the transfer rate. The oxygen supply device 94 may be operatively connected to the valves 82 which can be implemented to control the rate at which oxygen is supplied to the patient 24a. Any patient care devices including the IV device 90, the ventilator 92, and the oxygen supply device 94 are preferably attached to or incorporated into the smart bed 14a such that there are fewer external restrictions (e.g., hoses or tubes) limiting bed motion. Therefore, the patient 24a can be conveniently transported without restriction while remaining in bed, and the patient 24a can also be continuously cared for during such transportation.

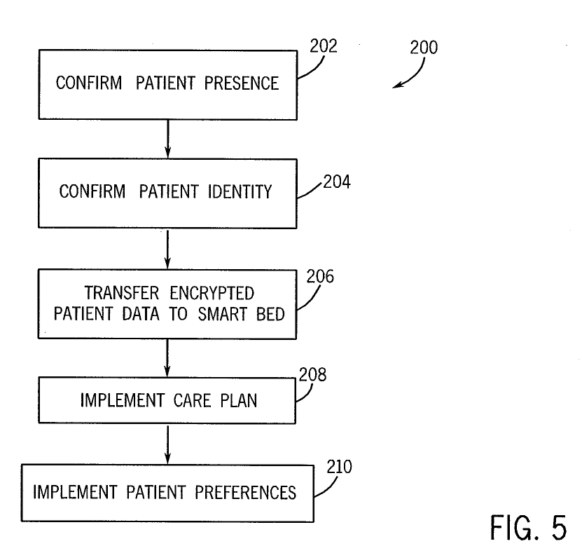

Referring to FIG. 5, a block diagram illustrating a method 200 for implementing the smart bed system 10 (shown in FIG. 1) is shown. The individual blocks of FIG. 5 represent steps that may be performed in accordance with the method 200.

At step 202, the method 200 confirms the presence of the patient 24a (shown in FIG. 1) within the smart bed 14a (shown in FIG. 1). This confirmation may be based on data from the RFID device 50 (shown in FIG. 3) or on feedback from the sensors 42 (shown in FIG. 3). In the embodiment wherein the RFID device 50 is implemented to confirm patient presence, there may be situations in which two or more patients 24a-24n (shown in FIG. 1) are close enough to a single smart bed 14a that the RFID antenna 40 (shown in FIG. 3) receives input from more than one RFID device 50. The smart bed 14a can be programmed to handle this situation in one of several ways. According to a first embodiment, the smart bed 14a is configured to recognize only the input from the first received RFID device and to ignore any input from subsequently received RFID devices. According to a second embodiment, the smart bed 14a is configured to recognize only the RFID device of a pre-selected patient, and to ignore any other RFID devices. According to a third embodiment, the smart bed 14a is configured to recognize only the RFID device that is closest to a predetermined location (e.g., the center of the smart bed 14a) and to ignore any other RFID devices. The third embodiment is particularly well adapted to distinguishing between the occupant of the smart bed 14a and another patients who may be passing by or visiting the occupant of the smart bed 14a.

At step 204, the method 200 confirms the identity of the patient 24a (shown in FIG. 1). The smart bed system 10 can implement one or more of the following exemplary methods for ensuring the patient 24a is properly identified. A first exemplary method for identifying the patient 24a includes downloading pre-recorded identification from the patient's RFID device 50 (shown in FIG. 3). A second exemplary method for identifying the patient 24a includes displaying a digital photographic image of the identified patient on the display 72 (shown in FIG. 4). Any hospital staff members attending to a particular patient can then compare the digital image with their actual patient to ensure the identification is accurate. Other known identification technology such as, for example, finger print, retinal scan, voice recognition, etc. can also be incorporated into the smart bed 14a (shown in FIG. 1) to help identify the patient 24a. As the patients can easily move from one smart bed to another, step 204 is an important precautionary measure adapted to insure that a patient and their data are properly correlated regardless of which smart bed the patient occupies.

According to one embodiment, if a given patient is determined to be in a smart bed that has been set up to receive another individual, the smart bed may be configured to initially deny service. Thereafter, a hospital staff member is alerted to determine who the patient is and where they should be. In this manner, the patient is prevented from potentially receiving treatment in accordance with another individual's care plan.

According to another embodiment, after the identity of the patient 24a (shown in FIG. 1) has been confirmed at step 204, the display 72 (shown in FIG. 4) may be configured to show any relevant patient data. “Relevant patient data” may, for example, include any patient data that is helpful in assessing patient progress or in determining an appropriate course of action. The patient data is preferably only shown when a caregiver such as the primary physician 16 (shown in FIG. 1) or the local caregiver 28 (shown in FIG. 3) is in sufficiently close proximity to treat the patient 24a. In this manner, the caregiver has access to the patient data needed to care for the patient 24a, and thereafter the data is removed from the display 72 in order to protect patient confidentiality.

At step 206, the method 200 transfers patient data such as, for example, the patient care plan 32 (shown in FIG. 1), medical history, medical reports, charts, patient preferences, etc. to the smart bed 14a (shown in FIG. 1). According to one embodiment, as a precautionary measure, all patient data is stored in an encrypted form on the server 12 (shown in FIG. 1). The encrypted patient data can only be read with a corresponding encryption key 86 (shown in FIG. 3) that may be included as part of the patient's RFID device 50 (shown in FIG. 3). Therefore, patient data for other patients cannot be read by the smart bed 14a such that the patient 14a is prevented from potentially receiving treatment in accordance with the wrong care plan.

At step 208, the smart bed 14a (shown in FIG. 1) implements the care plan 32 (shown in FIG. 1) that was downloaded at step 206. Implementation of the care plan 32 may include, for example, monitoring the patient 14a (e.g., with the sensors 42 (shown in FIG. 3)), collecting data from the sensors 42, transmitting collected data, caring for the patients 14a (e.g., with the output devices 70 (shown in FIG. 4)), etc. At step 210, the smart bed 14a implements any patient preferences that were downloaded at step 206. Implementation of any patient preferences may include, for example, adjusting the bed position and/or temperature in a preferred manner, providing a preferred entertainment media via the display 72 and/or speakers 74 (shown in FIG. 4), etc.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A smart bed adapted to accommodate a patient, said smart bed comprising:
a smart bed computer; and
a patient monitoring device operatively connected to the smart bed computer;
wherein the patient monitoring device is incorporated with the smart bed such that the smart bed is not restricted by any wires adapted to couple the patient monitoring device with the patient.

2. The smart bed of claim 1, wherein said patient monitoring device is selected from the group consisting of an electrocardiograph device, a pulse oximeter device, a blood-pressure monitoring device, an electromyogram device, and an electroencephogram device.

3. The smart bed of claim 1, further comprising a plurality of patient sensors operatively connectable to the patient monitoring device.

4. The smart bed of claim 1, wherein said patient sensors include sensors selected from the group consisting of motion sensors, thermal sensors, and pressure sensors.

5. The smart bed of claim 1, further comprising a bed monitoring device operatively connected to the smart bed computer.

6. The smart bed of claim 5, farther comprising a plurality of bed sensors operatively connected to the bed monitoring device.

7. The smart bed of claim 1, further comprising a wireless input device operatively connected to the smart bed computer.

8. The smart bed of claim 1, further comprising an input device operatively connected to the smart bed computer, said input device selected from the group consisting of a touch screen, a touch pad, a keyboard, a mouse, a joystick, a remote control, a track ball, and a Marquette trim knob.

9. A smart bed adapted to accommodate a patient, said smart bed comprising:
a smart bed computer;
a patient monitoring device operatively connected to the smart bed computer;
a plurality of patient sensors operatively connected to the patient monitoring device;
a bed monitoring device operatively connected to the smart bed computer;
a plurality of bed sensors operatively connected to the bed monitoring device;
wherein the patient monitoring device is incorporated with the smart bed such that the smart bed is not restricted by any wires adapted to couple the patient monitoring device with the plurality of patient sensors.

10. The smart bed of claim 9, wherein said patient monitoring device is selected from the group consisting of an electrocardiograph device, a pulse oximeter device, a blood-pressure monitoring device, an electromyogram device, and an electroencephogram device.

11. The smart bed of claim 9, wherein said plurality of patient sensors include sensors selected from the group consisting of motion sensors, thermal sensors, and pressure sensors.

12. The smart bed of claim 9, wherein said plurality of bed sensors include sensors selected from the group consisting of pressure sensors, mass sensors, and conductance sensors.

13. The smart bed of claim 9, further comprising a wireless input device operatively connected to the smart bed computer.

14. The smart bed of claim 9, further comprising an input device operatively connected to the smart bed computer, said input device selected from the group consisting of a touch screen, a touch pad, a keyboard, a mouse, a joystick, a remote control, a track ball, and a Marquette trim knob.

15. A smart bed system comprising:
a smart bed adapted to accommodate a patient, said smart bed including:
a smart bed computer; and
a patient monitoring device operatively connected to the smart bed computer, wherein the patient monitoring device is incorporated with the smart bed such that the smart bed is not restricted by any wires adapted to couple the patient monitoring device with the patient; and
a server coupled with the smart bed computer, said server configured to store any patient data pertaining to said patient, wherein said patient data can be selectively transferred from the server to the smart bed computer such that said patient data is accessible directly from the smart bed.

16. The smart bed system of claim 15, wherein said patient monitoring device is selected from the group consisting of an electrocardiograph device, a pulse oximeter device, a blood-pressure monitoring device, an electromyogram device, and an electroencephogram device.

17. The smart bed system of claim 15, wherein said smart bed further includes a plurality of patient sensors operatively connectable to the patient monitoring device.

18. The smart bed system of claim 15, wherein said smart bed further includes a bed monitoring device operatively connected to the smart bed computer; and a plurality of bed sensors operatively connected to the bed monitoring device.

19. The smart bed system of claim 15, wherein said smart bed further includes a wireless input device operatively connected to the smart bed computer.

20. The smart bed system of claim 15, wherein said smart bed further includes an input device operatively connected to the smart bed computer, said input device selected from the group consisting of a touch screen, a touch pad, a keyboard, a mouse, and a joystick, a remote control, a track ball, and a Marquette trim knob.
